(19) United States
(12) Patent Application Publication
(10) Pub. No.: US 2010/0036505 A1
(43) Pub. Date: Feb. 11, 2010

(54) PROSTHETIC DEVICE

(75) Inventor: Andreas Hassler, Rohrdorf (DE)

Correspondence Address: BLAKELY SOKOLOFF TAYLOR & ZAFMAN LLP, 1279 OAKMEAD PARKWAY, SUNNYVALE, CA, 94085-4040, US

(73) Assignee: Oped AG, Steinhausen (CH)

(21) Appl. No.: 11/721,535

(22) PCT Filed: Dec. 22, 2005
(86) PCT No.: PCT/EP2005/013944

§ 371 Date: Oct. 20, 2009

(30) Foreign Application Priority Data
Dec. 22, 2004 (DE) 102004063055.0

(51) Int. Cl. A61F 2/78

(52) U.S. Cl. 623/33

(57) ABSTRACT

A prosthetic device (10, 20, 31) having a shell arrangement for receiving an amputation stump and a frame mechanism (13) for connecting the shell arrangement to a prosthesis (16) is described, whereby the shell arrangement has at least two shell parts (11, 12; 21, 22; 32) that are arranged, opposite one another and enclose a receptacle space, the shell parts being joined together via the frame mechanism and their relative arrangement being variable, and the shell arrangement is provided at least partially with an evacuable lining (45) having a plurality of form particles (46) and having a fastening mechanism (25, 51) for securing the relative arrangement of the shell parts.

The present invention relates to a prosthetic device having a shell arrangement for receiving an amputation stump and a frame mechanism for connecting the shell arrangement to a prosthesis according to Claim 1.

Prosthetic devices of the type defined in the introduction are used for connecting a prosthesis to an amputation stump formed by amputation of an upper or lower extremity. In the case of amputation of a lower extremity, e.g., the leg below the knee joint, the prosthetic device may be used for secure connection of a prosthesis such as a leg support to the remaining stump of the lower leg. In the case of an amputation in the forearm area beneath the elbow joint, such a prosthetic device may be used for secure connection of a prosthetic gripping device that replaces the hand functions.

Such prosthetic devices that allow mechanical connection of the prostheses to the respective joint stump are usually connected to the body part in question only after “maturing of the stump” is concluded. In the past, following the surgical procedure in which the actual amputation is performed, so-called “interim prostheses” have been used; these are designed to be adaptable to the changing circumference of the amputation stump as the wound heals. Such interim prostheses serve mainly to support the maturing of a stump by promoting the detumescence process by applying a circumferential pressure that is adapted to the respective circumference of the stump.

The object of the present invention is to provide a prosthetic device of the type defined in the introduction that can be used during maturing of the stump and also after conclusion of maturing of the stump for connection of a functional prosthesis.

This object is achieved by a prosthetic device having the features of Claim 1.

With the inventive prosthetic device having a shell arrangement for receiving an amputation stump and a frame mechanism for connecting the shell arrangement to a prosthesis, the shell arrangement comprises at least two oppositely arranged shell parts enclosing a receptacle space, connected to one another via the frame mechanism and being variable in their relative arrangement. The shell arrangement of the inventive prosthetic device is provided at least partially with an evacuable lining that contains a plurality of form particles and is provided with a fastening mechanism for securing the relative arrangement of the shell parts.

Owing to the combination of the shell arrangement having a variable receptacle volume with the evacuable lining which allows the amputation stump to be accommodated in the shell arrangement in a manner that is always form-fitting, regardless of the prevailing volume of the stump at the moment, the circumferential pressure beneficial for the healing process can always be exerted without developing pressure points on the amputation stump due to the fact that the shell cooperates with the fastening device, which permits fixation of the shell parts in relation to one another. This ideally supports maturing of the stump.

In addition, the shell arrangement, in cooperation with the evacuable lining, also offers a possibility of creating a means of transfer of force over essentially the entire stump surface from the amputation stump to a prosthesis, i.e., a leg support, that can be connected to the frame mechanism.

Therefore, the inventive prosthetic device can advantageously be used to support maturing of the stump in the same way as for connection of a functional prosthesis to the amputation stump.

In an advantageous embodiment of the prosthetic device, the shell parts are connected to the frame mechanism in such a way that both the inside distance d_{S} of the shell parts in relation to one another as well as the shell opening angle α_{S} formed between the longitudinal axes of the shell parts are variable. First, this permits adaptation of the shell configuration to the amputation stump to a particular extent, but secondly, this also makes is possible to open the shell arrangement as far as possible for the operation of applying the prosthetic device to the amputation stump to thereby facilitate the application process.

If the shell parts have been designed with shape elasticity and have shell edges running in the longitudinal direction, their distance d_{W} defining the shell width being variable, then an adjustment of the shape of the shell arrangement can be undertaken.

The shape adjustment of the prosthetic device can be facilitated in particular by the fact that, for varying the shell width d_{W} of the shell parts, the frame mechanism has two frame legs, whose distance d_{R} in relation to one another is variable and which are connected to one another via a common frame base, each being connected to one of the two opposing shell edges of the shell parts.

If, in addition, the shell parts have elongated holes for joining to the frame legs of the frame mechanism, then the shell parts are also variable in their positioning with respect, to the frame legs.

In a preferred embodiment of the prosthetic device, the shell part (extensor-sided shell part) arranged on the extensor side next to the knee or elbow joint is rigidly connected to the frame legs, while the shell part arranged on the flexor side (flexor-sided shell part) is pivotably mounted on the frame legs or vice versa.

When the extensor-sided shell part has a recess in the knee or elbow area, a simplified adaptation to the joint area is possible.

When the extensor-sided shell part extends beyond the knee or elbow area, the extensor-sided shell, part may also be used for forced extension of the extremity.

However, as an alternative to the present design, of the extensor-sided shell part extending beyond the joint it is also possible for the extensor-sided shell part to extend at its proximal end into the area of the knee and for the flexor-sided shell part to extend at its proximal end beyond the area of the knee or elbow.

It is advantageous if the flexor-sided shell part has two shell straps on its proximal end, these straps being provided with a fastening mechanism for securing the relative arrangement of the shell straps. This allows the shell arrangement to be surrounded above the joint area as well.

As an alternative to a design of the flexor-sided shell part beyond the joint, it is also possible for the extensor-sided shell part to extend at its proximal end into the area of the knee or elbow and to be provided with a rail module that extends with its proximal end beyond the area of the knee or elbow and is provided at its proximal end with a fastening mechanism.

For adaptation of the prosthetic device to the extremity beyond the joint itself, it is advantageous if the rail module is provided with an articular mechanism in the area of the knee or the elbow.

To allow passive mobilization of the extremity in a defined flexor position or to allow a defined range of movement or flexion, the articular mechanism may be provided with an adjustment mechanism for adjusting a defined flexor angle and/or a defined range of flexion.

Preferred embodiments of the prosthetic device are explained in greater detail below with reference to the drawing, in which:

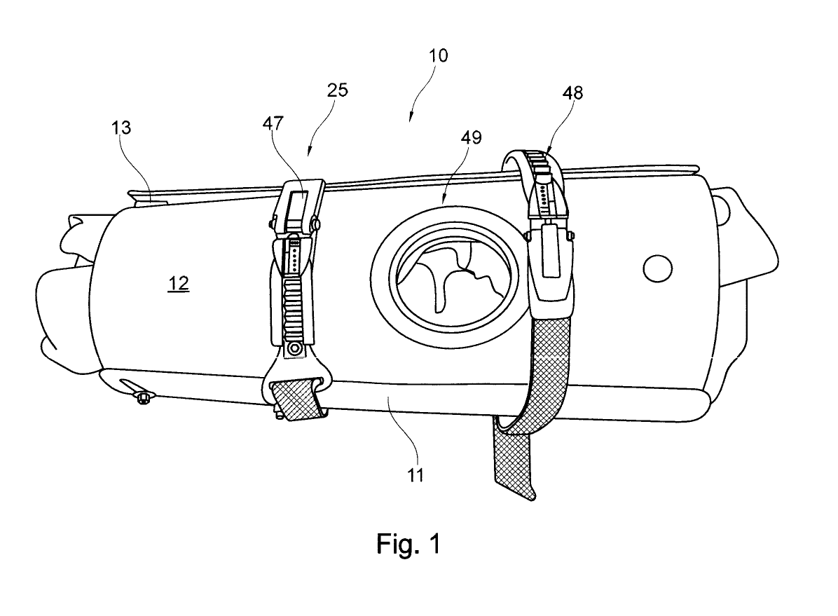

FIG. 1: shows a first embodiment of a prosthetic device in a view from above;

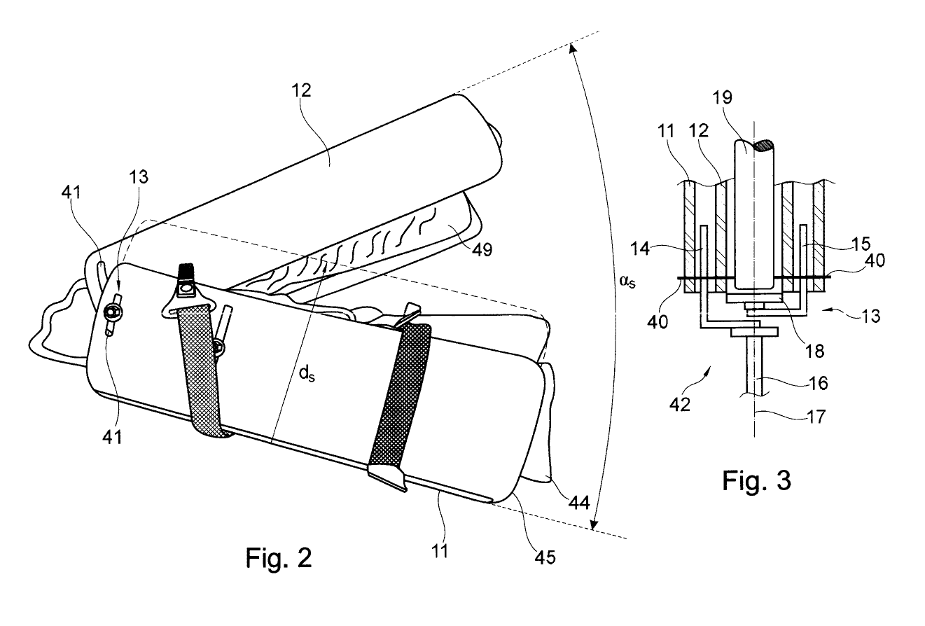

FIG. 2: shows the prosthetic device shown in FIG. 1 in a side view with the shell arrangement opened;

FIG. 3: shows a frame mechanism of a prosthetic device having a connected supporting part;

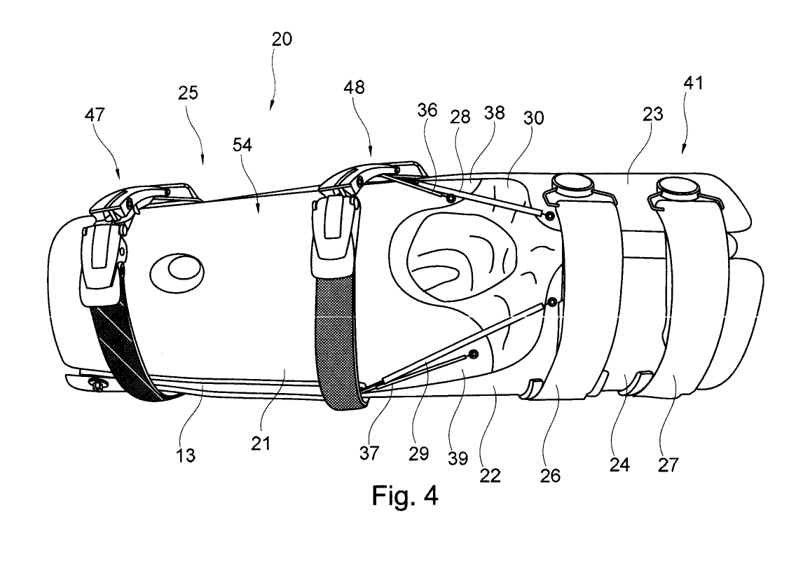

FIG. 4: shows a second embodiment of a prosthetic device in a view from above;

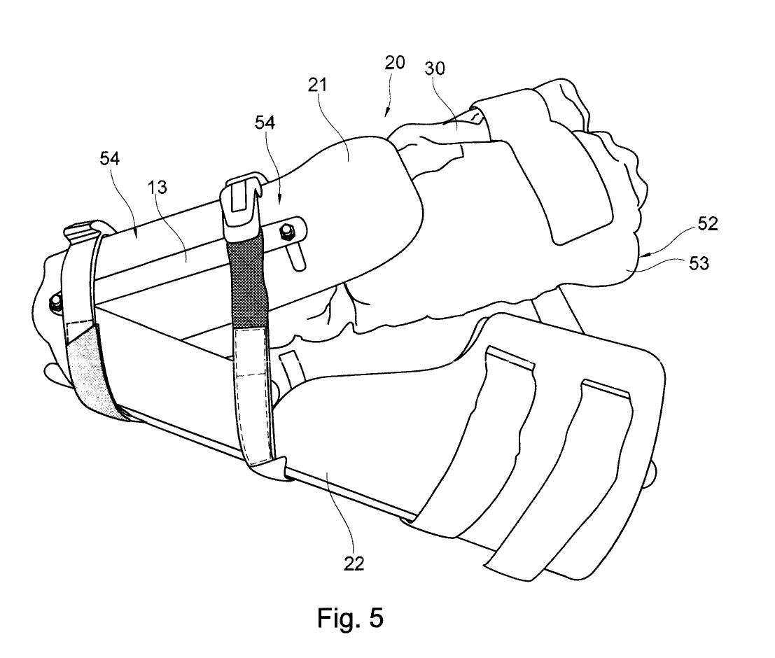

FIG. 5: shows the prosthetic device shown in FIG. 4 in a side view with the shell, arrangement opened;

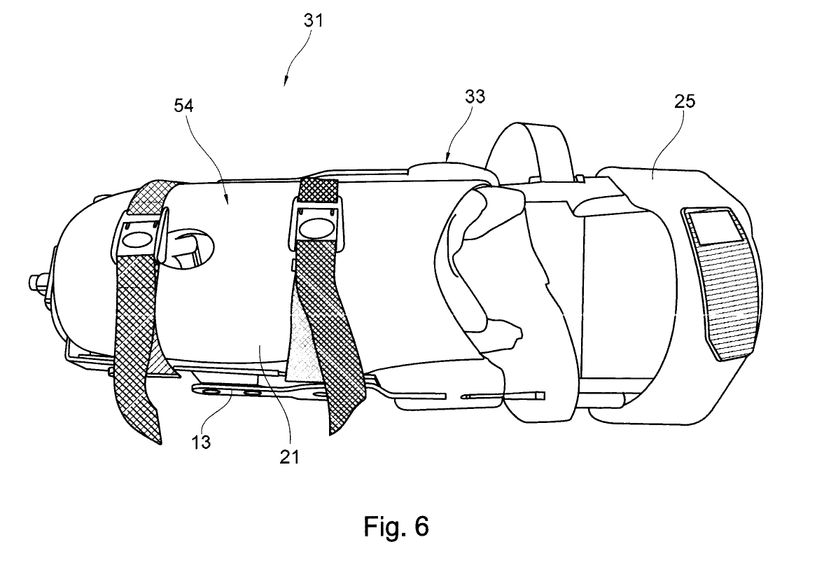

FIG. 6: shows a third embodiment of a prosthetic device in a view from above;

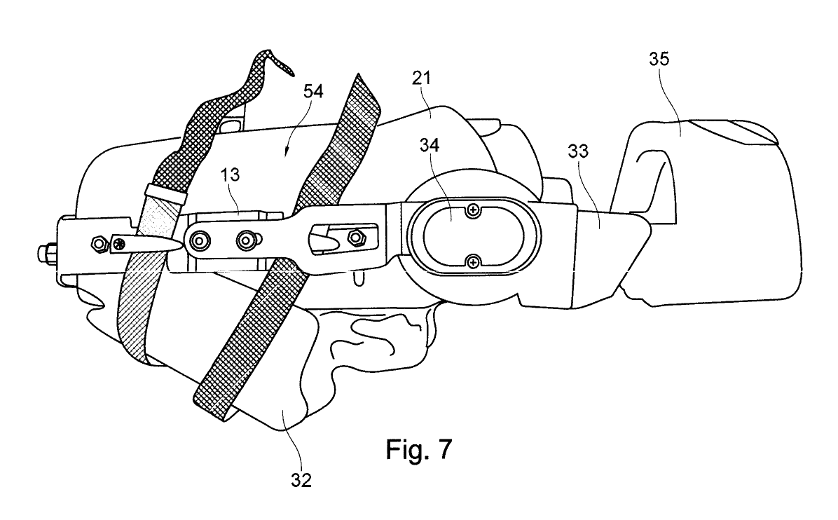

FIG. 7: shows the prosthetic device shown in FIG. 6 in a side view with the shell arrangement opened.

FIG. 1 shows a first embodiment of a prosthetic device 10 having a fibular shell 11 and a tibial shell 12 in a closed state in which the fibular shell 11 and the tibial shell 12 are connected to one another and secured by a fastening mechanism 25 formed by two tension belts 47, 48. The tibial shell 12 has a patellar opening 49 to receive a patella (not shown here) in the case of an amputation stump on the lower leg, inserted into the prosthetic device 10.

As shown by the opened or insertion position of the prosthetic device 10, as illustrated by FIG. 2, the fibular shell 11 and the tibial shell 12 are each connected at their distal ends to a frame mechanism 13, which is shown in a side view in FIG. 3, The frame mechanism 13 has two frame legs 14, 15, designed here in the form of an L, connected to a prosthesis, only a supporting part 16 of which is illustrated in FIG. 3, in the area of its leg base 42. As shown in FIG. 3, the two frame legs 14 and 15 are displaceable radially with respect to one another, i.e., in relation to a vertical axis 17. In the present, exemplary embodiment; the overlapping parts of the frame legs 14, 15 form a base 42 to which the supporting part 16 is connected. The frame legs 14, 15 are in contact with the outside wall of the tibial shell 12 on their ins ides and in contact with the inside wall of the fibular shell 11 on their outsides and are attached to these by bolt connections 40 so they can pivot relative to one another and/or are interconnected so they can be displaced against each other by means of the elongated holes 41 shown in FIG. 2. A cushion 18 for interim arrangement between the base 42 and an amputation, stump 19 is provided on the base 42.

In deviation from the depiction in FIG. 3, the frame legs 14, 15 of the frame mechanism 13 can be designed to bend about a vertical axis 17 of the prosthesis with respect to the base 42 thereof which serves to connect a prosthesis 16. In this way, an adjustment of the center of gravity of the body and/or the dynamic center of gravity that is established in walking can be adjusted advantageously in relation to the prosthesis tread area so that, for example, unnecessary loads on the stump due to transverse forces occurring can be minimized.

As shown to FIG. 2 in particular, a vacuum cushion 43 and/or 44 is arranged in the tibial shell 12 and in the fibular shell 11, the vacuum cushion being provided with an airtight outer shell 45 and a plurality of form particles arranged therein but not shown in detail here. In addition, the vacuum cushions 43, 44 have valve mechanisms (not shown in detail here) by means of which evacuation of the vacuum, cushions 43, 44 can be implemented.

In the operation configuration shown in FIG. 2, the prosthetic device 10 is prepared for insertion, i.e., placement of the amputation stump 19. After this insertion, the fibular shell 11 and the tibial shell 12 are swiveled toward one another for a form-fitting contact with the stump 19 and are secured in their relative arrangement with the fastening device 25. Then by evacuation of the vacuum cushions 43, 44 a form-fitting adjustment to the contour of the amputation stump 19 is achieved so that the molded body filling of the vacuum cushions 43, 44 is in supportive contact with the amputation stump 19. By means of the form-fitting adjustment, maturing of the stump is supported by a compression effect on the stump; furthermore, the form-fitting adjustment, which also allows a form-fitting connection, in the knee area due to the patellar disc opening 49 illustrated in FIG. 1, also provides supports in the direction of the load, so that reactive forces in walking are absorbed over the area by the prosthetic device 10 in the area of the vacuum cushions 43, 44.

As shown by a review of FIGS. 2 and 3 together, the shell parts 11, 12 are connected to the frame mechanism 13 in such a way that both the inside distance d_{S} and the shell opening angle α_{S} are variable. To vary the shell width d_{W} of the shell parts 11, 12, the two frame legs 14, 15 are variable in their distance d_{R} from one another and each is connected to the shell edges 55, 56.

FIGS. 4 and 5 show a prosthetic device 20 which has a lengthened fibular shell 22 in comparison with a tibial shell 21. The fibular shell 22 is constructed so that it has thigh straps 23, 24 extending toward one another above a knee area 50 formed in the tibial shell 21; these thigh straps having a proximal fastening device 51 formed here from two girdle belts 26, 27 in addition to the fastening mechanism 25, which has two tension belts 47, 48 that assume the role of fixation of the shell parts 21 and 22 in the stump area; this proximal fastening device 51 ensures a force-fitting connection of the thigh straps 23, 24 above the knee. As indicated in FIG. 4, the frame legs 14, 15 of the frame mechanism 13 may be connected to the thigh straps 23, 24 via pressure rods 28, 29 to support the contact of the thigh straps 23, 24 with the thigh above the knee. As an alternative or in addition, pressure bars 36, 37 may also be provided, acting on the knee straps 38, 39 of the tibial shell 21 that defines the knee area 50 to induce a close contact of the knee straps 38, 39 supporting the form-fitting connection beneath the knee.

As shown in FIG. 5, a vacuum cushion 30, which is provided in the prosthetic device 20, is designed in one piece and is arranged in such a way that free cushion edges 52, 53 that are opposite one another come to lie in the fibular area.

According to another embodiment. FIGS. 6 and 7 show a prosthetic device 31, which is provided with a tibial shell 21, in agreement with the prosthetic device 20 illustrated in FIGS. 4 and 5. In contrast with the prosthetic device 20, the prosthetic device 31 has a relatively short fibular shell 32 (FIG. 7). Instead, however, the tibial shell 21 is provided with a rail module 33 by means of the frame mechanism 13, said rail module being provided with a lockable articular mechanism 34 and a thigh holder 35 for attaching the shell to the thigh. The rail module 33 may be used as a passive mobilization rail, i.e., to achieve a forced extension in a manner similar to that of the fibular shell 22 of the prosthetic device 20 illustrated in FIGS. 4 and 5. However, defined forced flexion angles may he defined on the basis of the articular mechanism 34 or optionally flexion areas in which pivoting into a fixed deflection angle range is possible may also be defined.

With the prosthetic devices 20 and 31, an assembly group formed from the frame mechanism 13 and the tibial shell 21 may also be regarded as a basic unit 54 which is modular, i.e., can be supplemented by the fibular shell 22 or the fibular shell 32 and the rail module 33.

1. A prosthetic device having a shell arrangement for receiving an amputation stump and a frame mechanism for connecting the shell arrangement to a prosthesis, whereby the shell arrangement comprises at least two shell parts that are arranged opposite one another and surround a receptacle space, these shell parts being connected to one another via the frame mechanism and being variable in their relative arrangement and the shell arrangement is provided at least partially with an evacuable lining that contains a plurality of form particles, and having a fastening mechanism for securing the relative arrangement of the shell parts.

2. The prosthetic device according to claim 1, characterized in that
the shell parts are connected to the frame mechanism in such a way that both the inside distance d_{S} of the shell parts is variable in relation to one another and the shell opening angle α_{S} formed between the longitudinal axes of the shell parts is variable.

3. The prosthetic device according to claim 1, characterized in that the shell parts are designed with shape elasticity and have shell edges running in the longitudinal direction, the inside spacing of which defining the shell width is variable.

4. The prosthetic device according to claim 3, characterized in that
to vary the shell width of the shell parts, the frame mechanism has two frame legs that are variable in their distance d_{R} in relation to one another and are joined together by a common frame base, and each is connected to one of two opposing longitudinal edges of the shell parts.

5. The prosthetic device according to claim 1, characterized in that
the shell parts have elongated holes for connecting to the frame legs of the frame mechanism.

6. The prosthetic device according to claim 5, characterized in that
the shell part arranged on the extensor side of the amputation stump formed next to the knee joint or the elbow joint (extensor-sided shell part) is rigidly connected to the frame legs and the shell part (flexor-sided shell part) arranged on a flexor side is pivotably arranged on the frame legs or vice versa.

7. The prosthetic device according to claim 1,
characterized in that
the extensor-sided shell part has a recess in a knee area or an elbow area.

8. The prosthetic device according to claim 7,
characterized in that
the extensor-sided shell part extends beyond the area of the knee or elbow.

9. The prosthetic device according to claim 1,
characterized in that
the extensor-sided shell part extends at its proximal end into the knee area, and the flexor-sided shell part extends with its proximal end beyond the area of the knee or elbow.

10. The prosthetic device according to claim 9,
characterized in that
the flexor-sided shell part has two shell straps at the proximal end, said shell straps reducing the size of the shell part opening and being provided with another fastening mechanism for securing the relative arrangement of the shell straps.

11. The prosthetic device according to claim 1,
characterised in that
the extensor-side shell part extends with its proximal end into the area of the knee or elbow and is provided with a rail module which extends at its proximal end beyond the area of the knee or elbow and is provided with a fastening mechanism at its proximal end.

12. The prosthetic device according to claim 11,
characterized in that
the shell module is provided with an articular mechanism in the area of the knee or elbow.

13. The prosthetic device according to claim 12,
characterized in that
the articular mechanism is provided with an adjustment mechanism for adjusting a defined flexor angle and/or defined range of flexion.
